(19) United States
(12) Patent Application Publication
(10) Pub. No.: US 2004/0091545 A1
(43) Pub. Date: May 13, 2004

(54) Solid polyunsaturated fatty acid compositions

(76) Inventors: Justin Slagel, Leavesden (GB); Alan Bennett, Chigwell (GB); Rochelle N. Bennett, Chigwell (GB)

Correspondence Address: Dewitt Ross & Stevens, Intellectual Property Department, US Bank Building, 8000 Excelsior Drive Suite 401, Madison, WI, 53717-1914, US

(21) Appl. No.: 10/433,813

(22) PCT Filed: Dec. 5, 2001
(86) PCT No.: PCT/GB01/05382

(30) Foreign Application Priority Data
Dec. 6, 2000 (GB) 0029775.4
Sep. 26, 2001 (GB) 0123157.0

(51) Int. Cl. A61K 9/50; A61K 31/202

(52) U.S. Cl. 424/499; 514/560; 424/501

(57) ABSTRACT

A particulate omega-3 polyunsaturated fatty acid composition suitable for use as a medicament or foodstuff additive is obtained by acid precipitation from a basic dispersion or solution comprising an omega-3 polyunsaturated fatty acid, salt or ester, metabolite or other pharmacologically acceptable derivative thereof and a polymeric pharmaceutical excipient which may be a polymethacrylate or a polysaccharide or ether, ester or other derivative thereof.

The present invention relates to solid compositions of polyunsaturated fatty acids, especially omega-3 polyunsaturated fatty acids and particularly eicosapentaenoic acid (“EPA”). More particularly, it provides a solid composition of EPA with a polymeric pharmaceutical excipient such as a polymethacrylate (methacrylic acid copolymer) or derivative thereof or a polysaccharide, preferably cellulose or derivatives thereof, especially cellulose acetate (CA), cellulose acetate phthalate (CAP), hydroxypropyl methylcellulose (HPMC), hydroxypropyl methylcellulose phthalate (HPMCP) and/or other single or multiple ester and/or ether derivatives of cellulose.

EPA and other omega-3 polyunsaturated fatty acids are of dietary importance to humans and have been reported as having biological effects, particularly in relation to cardiovascular and inflammatory conditions. EPA is often poorly tolerated because of its unpleasant odour, taste and gastrointestinal side effects. EPA is a naturally occurring polyunsaturated fatty acid found in high concentration in fish oil.

CA, CAP, HPMCP and HPMC are among a number of cellulose derivatives widely used in oral pharmaceutical formulations, for example, as enteric coatings, films or binders for tablets or granules.

Polymethacrylates (methacrylic acid copolymers) are copolymers of methacrylic acid and an acrylic or methacrylic ester. They are widely used in oral capsule and tablet formulations and can produce a range of solubility characteristics depending on the particular polymethacrylate used.

JP-A-60204739 discloses a powder, containing lecithin, an eicosapentaenoic acid-based compound and cyclodextrin, for use as a medicine and health food supplement. The powder is prepared by dissolving the lecithin in the eicosapentaenoic-based compound by heating, followed by addition of the cyclodextrin and cooling of the resultant mixture.

JP-A-9087656 discloses a powdered fat obtained by coating a fat, containing e.g. EPA, with pullulan, which is a water-soluble polysaccharide cultured from the black yeast fungus Aureobasidium Pullulans.

JP-A-4178348 discloses a powdery clathrate compound of EPA or lower alkyl esters thereof with branched cyclodextrin to afford a water-soluble, highly stable additive for medicines or food.

EP-A-0289204 discloses disinfectant and pharmaceutical compositions comprising the lithium salt of C₁₈-C₂₂ polyunsaturated fatty acids. Specified polyunsaturated fatty acids include EPA. The pharmaceutical compositions can be for enteral, parenteral and topical administration and are for use in the treatment of conditions responsive to lithium and/or polyunsaturated fatty acid therapy. Conditions specified as being responsive to fatty acid therapy include Crohn's disease and ulcerative colitis. Reference is made to providing an enteric coating of, for example, an acrylate or cellulose acetate phthalate to delay release of the salt until it reaches the intestine.

U.S. Pat. No. 4,211,865 relates to therapeutically useful polymeric compositions of prostaglandin precursors. The polymers disclosed consist of a macromolecular polysaccharide matrix at least partly esterified by activated polyunsaturated acids containing 20 carbon atoms. Suitable polysaccharides specified include starch and dextrins, but not cellulose. Specified polyunsaturated acids include eicosapentaenoic acid.

U.S. Pat. No. 5,589,577 discloses the preparation of fatty acid esters of polysaccharides, suitable for use as coatings for the controlled release of active ingredients, especially fertilizers. Celluloses are specified as suitable polysaccharides of the invention although no specific fatty acids are identified.

EP-A-0336662 discloses the microencapsulation of fish oil within an enteric coating to provide a stable, odourless and tasteless composition for incorporation into a food product to reduce plasma triglyceride, low density lipoprotein and cholesterol levels. The specified coating materials are ethyl cellulose, cellulose acetate phthalate and cellulose acetate trimelitate. The microencapsulated fish oil formulation is prepared by atomizing an emulsion of, for example, 20 parts fish oil and 80 parts of a 25% suspension of ethyl cellulose in ammonium hydroxide into stirred glacial acetic acid in water (50:1000). The resultant is filtered and dried to give a formulation with a butter or cream cheese texture, depending on the dimensions of the atomized particles.

Zaniboni et al (Int. J. Pharmaceutics, 125, 151-155, 1995) discloses a method of preparing enteric beads of HPMCP. Specific reference is made to the incorporation of riboflavin (pKa 10.2; pKb 1.7) and riboflavin 5-phosphate into HPMCP beads. The beads were prepared by dropwise addition of a solution of HPMCP and sodium bicarbonate to a stirred citric acid solution.

U.S. Pat. No. 6,164,825 discloses a method of microencapsulating an oleophilic substance such as vitamin E to form a free flowing powder, by preparing an emulsion of the vitamin in an aqueous solution of a polymer such as methylcellulose and heat-setting the emulsion and drying by known methods such as spray-drying. A typical concentration of oleophilic substance in the composition disclosed is in the range 30-90% w/w of the composition.

It has now surprisingly been found that a substantially odourless powder or granular composition precipitates on addition of an acid to a basic dispersion or solution of an omega-3 polyunsaturated fatty acid (e.g. eicosapentaenoic acid), salt, ester or other pharmacologically acceptable derivative thereof and a polymethacrylate or a cellulose derivative (e.g. CA, CAP, HPMCP or HPMC or other single or multiple ester and/or ether derivative of cellulose).

Accordingly, in a first aspect of the invention, there is provided a particulate composition obtainable by acid precipitation from a basic dispersion or solution comprising an omega-3 polyunsaturated fatty acid, salt or ester, or pharmacologically acceptable metabolite or other derivative thereof and a polymeric pharmaceutical excipient selected from polymethacrylates, polysaccharides and ethers, esters or other derivatives of a polymethacrylate or polysaccharide.

The identity of the product is presently unknown, but may be, for example, an ester of the fatty acid with the polymeric pharmaceutical excipient. Alternatively, the free fatty acid or free ester (such as the ethyl ester) may be otherwise trapped in the polymeric pharmaceutical excipient.

In a second aspect of the invention, there is provided a process of preparing a particulate composition wherein a basic dispersion or solution comprising an omega-3 polyunsaturated fatty acid, salt or ester, or pharmacologically acceptable metabolite or other derivative thereof and a polymeric pharmaceutical excipient selected from polymethacrylates, polysaccharides and ethers, esters or other derivatives of a polymethacrylate or a polysaccharide is acidified and the resulting precipitate collected and dried.

In a third aspect of the invention, there is provided a particulate composition as defined above for use in a method of treating the human or animal body by way of diagnosis or therapy.

In a fourth aspect of the invention, there is provided a use of a particulate composition as defined above in the manufacture of a medicament for the treatment of inflammatory bowel disease.

The particulate composition may be in a powder or granular form.

The omega-3 polyunsaturated fatty acid may be, for example, linolenic acid, docosahexaenoic acid, eicosapentaenoic acid (EPA) or other C₁₈-C₂₂ fatty acid and preferably is a naturally occurring acid with 20 carbon atoms and more preferably is EPA. Salts and esters (such as the ethyl ester) of the omega-3 polyunsaturated fatty acid are included.

The omega-3 polyunsaturated fatty acid metabolite may be a simple metabolite formed, for example by enzyme hydrolysis of the fatty acid, or may be a more complex metabolite, for example a prostaglandin or other metabolite of fatty acids.

By pharmacologically acceptable metabolite or derivative of an omega-3 fatty acid, we mean to include any metabolite or derivative which has the same type of pharmacological activity as the parent omega-3 fatty acid.

Preferably, in all aspects of the invention, the particulate composition is obtainable by acid precipitation from a basic dispersion or solution comprising an omega-3 polyunsaturated fatty acid, salt or ester thereof and a polymeric pharmaceutical excipient as further defined herein.

More preferably, the particulate composition is obtainable by acid precipitation from a basic dispersion or solution comprising EPA or a salt thereof and a polymeric pharmaceutical excipient.

The polymeric pharmaceutical excipient is selected from the group consisting of polymethacrylates, polysaccharides and ethers, esters or other derivatives of a polymethacrylate or a polysaccharide. Suitable polymeric pharmaceutical excipients are those that, in the particulate form of the present invention, disintegrate at a point in the gastrointestinal tract beyond the stomach such that undesirable side effects associated with polyunsaturated fatty acids are avoided.

The polysaccharide is preferably a cellulose derivative. The cellulose may be any suitable single or multiple ether and/or ester of cellulose, for example, those derivatives of cellulose that are commonly used in enteral formulations, but is preferably CA, CAP, HPMCAS (hydroxypropyl methylcellulose acetate succinate), HPMCP, HPMC or a mixture thereof and more preferably HPMCP, HPMC or a mixture thereof.

By single or multiple ester and/or ether derivatives of cellulose, as used herein, we mean to include those derivatives of cellulose in which at least some of the hydroxy groups of cellulose are replaced by one or more (different) etheral groups and/or one or more (different) ester groups. An example of a multiple ether and multiple ester derivative of cellulose is HPMCAS (hydroxypropyl methylcellulose acetate succinate), where propyl and methyl ethers and acetyl and succinyl esters are incorporated.

The polymethacrylate may be a methacrylic acid copolymer which consists of copolymer of methacrylic acid and an acrylic or methacrylic ester in various ratios or may be an ammonium methacrylate copolymer. Ammonium methacrylate copolymers which could be used in the present invention include copolymers synthesized from acrylic acid and methacrylic acid esters with 5-10% of functional quaternary ammonium groups. Such ammonium methacrylate copolymers are marketed under the trade names Eudragit™ RL and Eudragit™ RS respectively. Suitable methacrylic acid copolymers include those with a ratio of free carboxyl groups to ester groups of 1:1, 1:2 or mixtures thereof, but preferably with a ratio of 1:1. Such methacrylic acid copolymers are marketed under the trade names Eudragit™ L (1:1) and Eudragit™ S (1:2). The most preferable methacrylic acid copolymer for use in the present invention is that marketed under the trade name Eudragit™ L 30 D-55, an aqueous dispersion or solution of a copolymer with a free carboxyl group to ester group ratio of 1:1, which dissolves above pH 5.5 and thus is insoluble in gastric media, but is soluble in the small intestine.

Other polymethacrylates suitable for use in the present invention include those marketed under the trade names Kollicoat™ 30D and Kollicoat™ 30DP.

Polysaccharides or polymethacrylates suitable for use in the present invention are preferably soluble in basic solutions and when in a basic solution, result in precipitation of the polysaccharide or polymethacrylate or product upon acidification.

More than one polymeric pharmaceutical excipient may be used in preparing a particulate composition of the present invention. If, for example two exicpients are utilized, preferably at least one is soluble in the basic solution and the second may or may not be.

Optionally, further components can be included in the formulation. A binder/diluent, for example microcrystalline cellulose, can be included. In a preferred embodiment of the invention, the formulation comprises a polysaccharide pharmaceutical excipient, such as an HPMC ester, and microcrystalline cellulose.

Additionally or alternatively, a granulation agent such as polyvinylpyrrolidone (PVP) or polyvinylpolypyrrolidone (PVPP) can be included in the formulation to enhance the granular quality or “flowability” of the particulate composition. In this regard, PVP (e.g. Povidone™) is particularly preferred.

In a particularly preferred embodiment of the present invention, the formulation comprises EPA or salt or ester (e.g. the ethyl ester) thereof, a pharmaceutical excipient being a cellulose derivative such as CA, CAP, HPMC, HPMCP and/or other single or multiple ester and/or ether of cellulose, a binder/diluent such as microcrystalline cellulose and an excipient to improve the “flowability” (a granulation agent) of the formulation such as PVP. The granulation agent is suitably incorporated into the formulation by standard methods after the particulate formulation of the invention has been formed.

The basic dispersion or solution may be prepared either by adding the fatty acid, salt, ester or other derivative thereof to a basic dispersion or solution (preferably a solution) of the polysaccharide or ether, ester or other pharmacologically acceptable derivative thereof or by adding the polymeric pharmaceutical excipient to a basic dispersion or solution of the fatty acid, salt, ester, metabolite or pharmacologically acceptable derivative thereof.

The basic dispersion or solution preferably comprises a weak base, for example an alkaline organic substance such as an amino acid (e.g. lysine) or an amine, an alkali metal (e.g. Li, Na, K) salt of a weak acid, an alkali metal hydroxide, an alkaline salt of a carbonic acid, phosphonic acid or silicic acid or an alkaline ammonium salt, and more preferably alkali metal salts of bicarbonate or carbonate (e.g. sodium or potassium bicarbonate). Preferably the basic solution is an aqueous sodium bicarbonate or carbonate solution.

The aqueous solution of sodium bicarbonate, or other base, may be a 0.5-5% w/v solution and preferably is 2% w/v solution.

The basic dispersion or solution may be acidified, for example by bubbling a suitable acidic gas through the basic solution or, preferably, by adding an acidic solution to it. Preferably, the acidic solution is a weak acid and more preferably is a mono-, di- or tri-carboxylic acid, especially a tricarboxylic acid such as citric acid.

The acidic solution, for example a citric acid solution, may be a 2-80% w/v solution, preferably 10-20% w/v solution and more preferably a 15% w/v solution.

In a preferred embodiment, the fatty acid, salt or ester, metabolite or other pharmacologically acceptable derivative thereof is added to a basic dispersion or, preferably, solution of the polymeric pharmaceutical excipient. The acidic solution is then added to the resulting mixture with stirring (preferably in a mechanical stirrer/mixer device, e.g. in a Silverson™ stirrer). The precipitate is then filtered and dried to give a powder. Preferably, the precipitate is freeze-dried (lyophilised).

In another embodiment, the polymeric pharmaceutical excipient is added to a basic dispersion or solution of the fatty acid, salt or ester, metabolite or other pharmacologically acceptable derivative thereof and the acidic solution added to the resulting mixture with stirring. The resulting precipitate is filtered and dried to give a granular solid or powder.

Suitably, 25 g to 200 g of polymeric pharmaceutical excipient (calculated as HPMCP) can be used per 100 ml fatty acid or derivative thereof (calculated as free acid). Preferably, about 50 g of polymeric pharmaceutical excipient (calculated as HPMPC) may suitably be used per 100 ml of fatty acid or derivative (calculated as free acid).

Preferably, the proportion of the fatty acid in the composition of the invention is greater than 40% by weight and more-preferably greater than 60% by weight.

Further specific embodiments of the present invention include the following procedures. An alkaline solution of HPMCP may be mixed with EPA to form a dispersion or solution, a polymethacrylate added to the mixture which is then stirred. An acid may then be added to the resultant (followed by vigorous stirring) to precipitate a particulate composition. Alternatively, an alkaline dispersion of EPA (preferably adjusted to about pH 8 or 9) may be treated with a polymethacrylate and the resulting mixture treated with acid (followed by stirring) to form a precipitate. In another embodiment, HPMC may be added to an alkaline solution of HPMCP to form a dispersion. EPA may then be added to the dispersion and the resultant mixture stirred and then treated with acid (followed by stirring) to form a precipitate. In a further alternative embodiment, a polymethacrylate may be added to an HPMCP solution and then EPA added to the resultant dispersion. Acid may then be added to the resultant mixture, with stirring, to form a precipitate.

The composition may be used in the treatment of a range of conditions including cancer (particularly prostate cancer, for example by reduction in levels of prostate-specific antigen (PSA)), asthma, pancreatitis, cardiovascular disorders and inflammatory disorders. Preferably, the composition is used in the treatment of inflammatory disorders, especially inflammatory bowel disease.

The composition may also be used for application to mucosal and other surfaces.

The compositions of the present invention are suitable for oral administration as medicaments and as foodstuff additives.

The compositions of the present invention provide orally administerable EPA with minimal undesirable odour and taste more normally associated with EPA and other fatty acids derived from fish oil.

The present invention also provides EPA-containing compositions that allow release of the EPA in the gut, in a pH-dependent manner, in order that it can evoke its therapeutic effect.

Preferred compositions of the present invention should not release significant amounts of its EPA content in the stomach, e.g. at pH of 1.2, preferably less than 5% and most preferably substantially zero. Preferred compositions should also release EPA in the intestine, preferably the upper small intestine, i.e. at pH in the region 5.5 to 7.5. It should release at least 50% of its EPA content in the intestine, preferably at least 70%, more preferably at least 90% and most preferably substantially all of its EPA content. Preferably at least 90% of its content is released within 3 hours of the composition of the invention reaching the small intestine and more preferably within 1 hour.

The compositions of the invention may be formulated as hard or soft gelatin capsules, compressed tablets, or provided in a form for sprinkling on food or adding to a drink such as orange juice, for example.

The invention will now be illustrated by the following non-limiting Examples.

### EXAMPLE 1

Hydroxypropyl methylcellulose phthalate (HPMCP) (20 g) was added to an aqueous sodium bicarbonate solution (2% w/v, 100 ml) and the mixture agitated to ensure that the HPMCP dissolved. EPA (40 ml) was added to the HPMCP solution and the resultant stirred for 15 minutes. Citric acid solution (15% w/v) was slowly added to the EPA/HPMCP/NaHCO₃ dispersion and the resulting mixture stirred for one hour. The mixture was then left to settle and the precipitate collected by filtration. The solid was freeze-dried to yield a powder.

### EXAMPLE 2

EPA (40 ml) was added to an aqueous sodium bicarbonate solution (2% w/v) and the mixture agitated for 15-30 minutes. HPMCP (20 g) was added to the EPA/sodium bicarbonate dispersion and the mixture agitated for 45-90 minutes. Citric acid solution (15% w/v) was slowly added to the EPA/HPMCP/NaHCO₃ dispersion and the resulting mixture stirred for one hour. The mixture was then left to settle and the precipitate collected by filtration. The solid was freeze-dried to yield a powder.

### EXAMPLES 3-9

Several further formulations were prepared according to the method of Example 1. Where HPMC or microcrystalline cellulose are included in the formulations, it should be noted that those ingredients were added to the HPMCP/sodium bicarbonate mixture prior to addition of EPA. Where polyvinylpyrrolidone (PVP, Povidone™) or sodium starch glycollate is included in the formulation, these ingredients were added after the precipitate was obtained.

The relative percentage content of each component in the formulations of Examples 3-9 is presented in Table 1.

TABLE 1
Percentage of each component in formulations of
Examples 3-9

Percentage of formulation content
for each Example

| Ingredient | 3 | 4 | 5 | 6 | 7 | 8 | 9 |
|---|---|---|---|---|---|---|---|
| EPA | 66 | 66 | 66 | 65 | 62 | 66 | 65 |
| HPMCP | 34 | 17 | 17 | 16.5 | 15.5 | 25.5 | 25 |
| HPMC | | 17 | | | | | |
| Microcrystalline | | | 17 | 16.5 | 15.5 | 8.5 | 8 |
| cellulose | | | | | | | |
| PVP ^{a} | | | | 2 | 2 | | 2 |
| Sodium Starch | | | | | 5 | | |
| Glycollate | | | | | | | |

^{a} PVP = polyvinylpyrrolidone (Povidone™)

The formulations of Examples 6 and 9 were of a granular powder, whereas the Examples 3-5, 7 and 8 were a more ‘fluffy’ powder.

### EXAMPLE 10

### Dissolution Study of EPA Powder

An in vitro simulation of certain in vivo conditions was adopted using the following method.

Approximately 1 g of the powdered formulation was placed in a mesh basket and placed into the vessel of a BP Apparatus II (paddle method) containing the medium (500 ml) of a desired pH and at a temperature of 37° C. Octan-1-ol (50 ml) was added and the paddles lowered and rotated at 50 rpm. An aliquot of the organic layer was removed after 1 hour and the sample analysed using a standardised gradient gas chromatography system with FID detection.

The results of the dissolution study at pH 1.2 (approximating to gastric conditions) and at pH 7.5 (approximating to conditions in the intestine) are presented in Table 2.

TABLE 2
Dissolution of EPA from formulations of Examples 3, 4 &5.

| | % EPA released after 1 h | | |
|---|---|---|---|
| PH of medium | Example 3 | Example 4 | Example 5 |
| 1.2 | <1 | 14 | 12 |
| 7.5 | 16 | 29 | 80 |

### EXAMPLE 11

### Dissolution Study of EPA Powder in Gelatin Capsule

A gelatin capsule containing the EPA formulation of Examples 3, 5, 6, 7, 8 or 9 was wrapped in stainless steel wire (to keep the capsule at the bottom of the dissolution vessel of the apparatus (BP Apparatus II—paddle method)) and placed in the medium of desired pH and at 37° C. Octan-1-ol was added and the paddles rotated. An aliquot was removed from the organic layer after 1 hour. The samples were analysed using a standardised gradient gas chromatography system with FID detection.

Table 3 shows the results of the assay using the formulation of Examples 3 and 5 in single capsule experiments.

Table 4 shows the results of the assay using the formulation of Examples 5-9 using the mean of 3 capsules with the standard deviation in brackets.

TABLE 3
Dissolution of EPA from gelatin capsule containing
formulation of Examples 3 & 5.

% EPA released after 1 h

| pH of medium | Example 3 | Example 5 |
|---|---|---|
| 1.2 | 11/10 | 18/7 |
| 7.5 | 66/65 | 100/98 |

Paddle speed = 50 rpm; in 500 ml of medium

TABLE 4
Dissolution of EPA from gelatin capsules
containing the formulation of Examples 5-9

| | % EPA release after 1 h | | | | |
|---|---|---|---|---|---|
| | - mean of 3 capsules (RSD) | | | | |
| pH | 5 | 6 | 7 | 8 | 9 |
| 1.2 | 2 | 4 | 5 | 3 | 2 |
| 5.5 | 32 (9.6) | 25 (10.1) | | | |
| 7.5 | 97 (6.6) | 70 (27.6) | 44 (4.5) | 70 (9.1) | 66 (9.2) |

1. A particulate composition obtainable by acid precipitation from a basic dispersion or solution comprising an omega-3 polyunsaturated fatty acid, salt or ester, or pharmacologically acceptable metabolite or other derivative thereof and a polymeric pharmaceutical excipient selected from polymethacrylates, polysaccharides and ethers, esters or other derivatives of a polymethacrylate or polysaccharide.

2. A particulate composition as claimed in claim 1; wherein the basic dispersion or solution comprises an omega-3 polyunsaturated fatty acid or a salt or ester thereof.

3. A particulate composition as claimed in claim 1 or claim 2, wherein the fatty acid is EPA.

4. A particulate composition as claimed in any one of the preceding claims, wherein the polysaccharide is selected from one or more of CA, CAP, HPMCAS, HPMCP and HPMC.

5. A particulate composition as claimed in claim 4, wherein the polysaccharide is selected from HPMCP and HPMC or is an admixture thereof.

6. A particulate composition as claimed in any one of the previous claims, which further comprises at least one of a binder/diluent and a granulation agent.

7. A particulate composition as claimed in any one of the previous claims wherein the weight/volume percent proportion of polymeric pharmaceutical excipient to fatty acid is in the range 25 to 200%.

8. A particulate composition as claimed in claim 7, herein the weight/volume percent is about 50%.

9. A process of preparing a particulate composition wherein a basic dispersion or solution comprising an omega-3 polyunsaturated fatty acid, salt or ester, or pharmacologically acceptable metabolite or other derivative thereof and a polymeric pharmaceutical excipient selected from polymethacrylates, polysaccharides and ethers, esters or other derivatives of a polymethacrylate or a polysaccharide is acidified and the resulting precipitate collected.

10. A process as claimed in claim 9, wherein the basic dispersion or solution comprises an omega-3 polyunsaturated fatty acid or a salt or ester thereof.

11. A process as claimed in claim 9 or claim 10, wherein the fatty acid is EPA.

12. A process as claimed in any one of claims 9 to 11, wherein the basic dispersion or solution is obtained by adding the fatty acid, salt or ester, or pharmacologically acceptable metabolite or other derivative thereof to a basic dispersion or solution of the polymeric pharmaceutical excipient.

13. A process as claimed in any one of claims 9 to 12, wherein the basic dispersion or solution is acidified by dropwise addition of an acidic solution.

14. A process as claimed in any one of claims 9 to 13, wherein the polysaccharide is selected from one or more of CA, CAP, HPMCAS, HPMCP and HPMC.

15. A process as claimed in claim 14, wherein the polysaccharide is selected from HPMCP and HPMC or is an admixture thereof.

16. A process as claimed in any one of claims 9 to 15, wherein the precipitate is lyophilised.

17. A particulate composition as claimed in any one of claims 1 to 8 for use in a method of treating the human or animal body by way of diagnosis or therapy.

18. Use of a particulate composition as claimed in any one of claims 1 to 8 in the manufacture of a medicament for the treatment of inflammatory bowel disease.

19. A method of treating inflammatory bowel disease comprising administering to a patient an effective amount of a particulate composition as claimed in any one of claims 1 to 8.
